(19) United States
(12) Patent Application Publication
(10) Pub. No.: US 2002/0004957 A1
(43) Pub. Date: Jan. 17, 2002

(54) ONE STEP METHOD AND COMPOSITIONS FOR SIMULTANEOUSLY COLORING AND HIGHLIGHTING HAIR

(76) Inventor: TERESITA VERGARA IMPERIAL, STATEN ISLAND, NY (US)

Correspondence Address: WARD AND OLIVA, 708 THIRD AVENUE, NEW YORK, NY, 10017, US

(21) Appl. No.: 09/332,863

(22) Filed: Jun. 15, 1999

(51) Int. Cl. A61K 7/13

(52) U.S. Cl. 8/405; 8/406; 8/408

(57) ABSTRACT

A single composition for simultaneous coloring and highlighting hair to provide hair fibers having variations in tonality, hue, and/or shade, comprising, 1-20% inorganic persulfate, 1-20% hydrogen peroxide, 0-10% of one or more cationic dye molecules; and 0.01-20% of one or more cationic surfactants; as well as a one step method for simultaneously color and highlighting hair to provide hair fibers having variations in tonality, hue, and/or shade comprising, combining, immediately prior to use, a powder composition comprised of at least one inorganic persulfate, an aqueous developer composition comprised of hydrogen peroxide; and an aqueous based colorant composition; and applying the mixture to the hair for a period of time sufficient to cause coloration and highlighting of the hair.

### TECHNICAL FIELD

The invention is in the field of coloring hair, including compositions and methods for use therein.

### BACKGROUND OF THE INVENTION

Hair color is used by a large percentage of the population. The reasons consumers may color hair vary from a desire to change hair color, cover grey hair, or provide unique highlighting effects. In addition, certain funky looks which are popular with young people include “streaking” or “chunking”, where different segments of the colored hair show substantial shade variations.

The traditional procedure for achieving streaked or chunked hair, as well as hair exhibiting more subtle variations in tonality and hue, comprises two steps. The hair is first bleached. The second step involves coloring the hair with the desired hair color. This two step process is disadvantageous because the bleach step often results in uneven removal of color from the hair and the hair is not colored in the desired shade. In addition, the process takes twice the time, and may require heat, thus may cause increased damage to overly sensitive hair as both the bleach and the colorant are most often highly alkaline, and are on the hair consecutively. This current method is particularly disadvantageous for African Americans who wish to chunk or highlight fragile hair that may already have been relaxed or straightened.

U.S. Pat. No. 5,688,291 teaches a single-step method and compositions for simultaneously lightening and coloring hair up to seven levels.

Japanese Patent Publication No. 08175940, published Jul. 9, 1997, teaches the combination of a xanthene-based hair dyeing agent, a peroxide based developer, and a persulfate based bleaching compound.

In general, there is a need for stable hair color compositions and methods which are capable of providing subtle to dramatic variations in tonality and hue at one extreme, and at the other extreme, subtle to pronounced variations in shade in a one step procedure. These hair color compositions must also be stable, gentle, and suitable for use with those who have sensitive or otherwise chemically treated hair. In particular, the compositions and methods must be suitable suitable for use on African American hair which has chemically treated with hair color, hair relaxer, or other straightening agents.

The object of the invention is to provide a one step method for simultaneously coloring and highlighting hair to provide hair having variations in tonality and hue, preferably without heat.

The object of the invention is to provide a one step method for simultaneously coloring and highlighting hair up to ten levels, e.g. ten shades lighter than the original shade.

Another object of the invention is to provide a one step method for coloring hair to provide hair with subtle to pronouced variations in shade.

Another object of the invention is to provide a one step method for coloring hair to provide hair colored in the desired shade without any off warm/orange tones.

Another object of the invention is to provide a one step method for coloring and highlighting African American hair, particularly hair which has already been treated with straightening agents, preferably using lower levels of persulfate than that used in traditional systems.

Another object of the invention is to provide a method for coloring and highlighting hair with semi-permanent dyes that provide the same degree of coloration as oxidation, or permanent, dyes.

Another object of the invention is to provide a single composition for coloring and highlighting hair without causing undue hair damage due to double processing.

Another object of the invention is to provide a hair color composition and one step coloring process that is capable, at one extreme, of providing subtle variations in tonality and hue and at the other extreme is capable of providing subtle to pronouced variations in hair shade.

Another object of the invention is to provide a one step hair color process and compositions which provide consistent color to the user's hair, e.g. the result obtained is not dependent on the color of the user's hair. This is in contrast to traditional hair color processes, where the final result depends on the color of the user's hair.

### SUMMARY OF THE INVENTION

The invention comprises a composition for simultaneously coloring and highlighting hair to provide hair fibers having variations in tonality, hue, and/or shade, comprising, by weight of the total composition:

(a) 1-20% inorganic persulfate,

(b) 1-20% hydrogen peroxide,

(c) 0-10% of at least one cationic dye molecule; and

(d) 0.01-20% of one or more cationic surfactants.

The invention also comprises a one step method for simultaneously coloring and highlighting hair to provide hair fibers having variations in tonality, hue and/or shade, comprising the steps of:

(a) combining, immediately prior to use, (i) a powder composition comprised of at least one inorganic persulfate, (ii) an aqueous developer composition comprised of hydrogen peroxide; and (iii) an aqueous based colorant composition comprised of 0 to 10% of at least one cationic dye; and

(b) applying the mixture of (a) to the hair for a period of time sufficient to cause coloration and highlighting of the hair.

### DETAILED DESCRIPTION

The invention comprises both a composition used to provide highlighting and coloration to hair as well as a one step method for coloring and highlighting hair. All percentages mentioned herein are percentages by weight unless otherwise indicated.

I. THE COMPOSITION

The composition for simultaneously coloring and highlighting hair to provide hair fibers having variations in tonality and hue, comprises about:

(a) 1-20% inorganic persulfate,

(b) 1-20% hydrogen peroxide,

(c) 0.01-10% of at least one cationic dye molecule; and

(d) 0.01-20% of one or more cationic surfactants.

The composition is prepared by mixing (i) a powder bleach composition, (ii) an aqueous developer composition; and (iii) an aqueous based colorant composition comprised of at least one cationic dye.

A. The Powder Bleach Composition

The powder bleach composition is generally a mixture of persulfate compounds which are capable of bleaching the hair, particulate fillers, and, if desired, inorganic particulate colorants.

1. Persulfates

The powder bleach composition comprises 15-65%, preferably 20-60%, more preferably 25-55% by weight of the total composition of one or more inorganic persulfates which may be alkali metal or alkaline earth metal persulfates, or ammonium persulfate. Preferably the persulfate comprises on or more of an alkali metal or alkaline earth metal persulfate. Examples of alkali metal persulfates include lithium, sodium, potassium, cesium, and the like. Examples of suitable alkaline earth metals include magnesium, calcium, and the like. Particularly preferred are sodium and potassium persulfates. The persulfates are generally in particulate form, have have particle sizes ranging from about 0.1 to 200 microns.

2. Particulate Fillers

The remainder of the powder bleach composition comprises particulate fillers. Preferably, the powder bleach composition comprises 5-60%, preferably 8-55%, more preferably 10-50% by weight of the total composition of the particulate fillers. The term “particulate filler” means a generally inert particulate having a particle size of about 0.1-250 microns. The particulate fillers provide volume and, when mixed with the persulfates, dilute the persulfate particles. A variety of particulate fillers are suitable including inorganics, inorganic salts, hydrophilic colloids, carbohydrates, soaps, alkyl sulfates, and the like.

(a) Inorganics

Examples of inorganics include silica, hydrated silica, alumina, attapulgite, bentonite, calcium oxide, chalk, diamond powder, diatomaceous earth, fuller's earth, hectorite, kaolin, mica, magnesium oxide, magnesium peroxide, montmorillonite, pumice, talc, tin oxide, zeolite, zinc oxide, and the like.

(b) Inorganic Salts

Examples of suitable inorganic salts include aluminum, sodium, potassium, and magnesium salts of inorganic or organic acids. Examples of suitable salts include sodium metasilicate, sodium chloride, sodium silicate, aluminum citrate, calcium saccharin, calcium salicylate, calcium citrate, calcium benzoate, magnesium acetate, magnesium ascorbate, magnesium PCA, magnesium gluconate, potassium acetate, potassium benzoate, potassium citrate, potassium sorbate, sodium acetate, sodium ascorbate, sodium citrate, sodium gluconate, sodium pyruvate, and mixtures thereof

(c) Hydrophilic Colloids

Examples of suitable hydrophilic colloids include hydroxyethylcellulose, locust bean gum, maltodextrin, methylcellulose, agar, dextran, dextran sulfate, gelatin, pectin, potassium alginate, sodium carboxymethylchitin, xanthan gum, and the like.

(d) Carbohydrates

Examples of suitable carbohydrates include sugars such as glucose, sucrose, maltose, xylose, trehelose, and derivatives thereof, in particular sugar esters of long chain, C₁₄₋₃₀ fatty acids, as well as dextrins, cellulosics, and derivatives thereof.

(e) Soaps and Alkyl Sulfates

Examples of soaps and alkyl sulfate particles that may act as particulate fillers include the aluminum, sodium, and potassium salts of fatty acids such as aluminum distearate, aluminum isostearate, aluminum myristate, calcium behenate, calcium stearate, calcium behenate, magnesium stearate, magnesium tallowate, potassium palmitate, potassium stearate, potassium oleate, sodium stearate, sodium oleate, sodium myristate, sodium palmitate, and the like. Suitable alkyl sulfates include sodium lauryl sulfate, sodium cetyl sulfate, sodium myristyl sulfate, sodium octyl sulfate, and the like.

3. Inorganic Colorants

If desired, the powder bleach composition may comprise 0.01-2%, preferably 0.05-1%, more preferably about 0.1-1% of an inorganic colorant. The inorganic colorant is preferably in the particulate form and will provide a subtle coloration to the powder composition to make it more aesthetically pleasing for commercial purposes. Particularly preferred for use in the bleach composition is ultramarine blue.

B. The Aqueous Developer Composition

The aqueous developer composition comprises, by weight of the total composition, 50-99% water, 1-30% hydrogen peroxide, and 0.01-30%, preferably 0.05-20%, more preferably 0.1-15% of an oily phase. The aqueous developer composition may be in the form of a water-in-oil or oil-in-water emulsion or clear aqueous solution.

1. Oily Phase Ingredients

Suitable oils are liquid at room temperature (25° C.) and include hydrocarbon oils and/or silicone oils which are volatile or nonvolatile, and glyceryl esters of fatty acids. The term “volatile” means that the oil has a measureable vapor pressure, i.e. a vapor pressure of at least 2 mm. of mercury at 20° C. The term “nonvolatile” means that the oil has a vapor pressure of less than 2 mm. of mercury at 20° C. Suitable volatile oils generally have a viscosity of 0.5 to 10 centistokes at 25° C., and include linear silicones, cyclic silicones, paraffinic hydrocarbons, or mixtures thereof

(a) Volatile Silicones

Cyclic silicones (or cyclomethicones) are of the general formula:

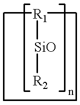

wherein R₁ and R₂ are each independently H, C₁₋₈ alkyl, aryl, aralkyl, alkenyl, or a cyclic or alicyclic ring, preferably a C₁₋₄ alkyl, most preferably methyl, and wherein n=3-7.

Linear volatile silicones in accordance with the invention have the general formula:

(CH₃)₃Si—O—[Si(CH₃)₂—O]ₙ—Si(CH₃)₃

where n=0-6, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

(b) Volatile Paraffinic Hydrocarbons

Also suitable as the volatile oils are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable. Transfer resistant cosmetic sticks of the invention will generally comprise a mixture of volatile silicones and volatile paraffinic hydrocarbons.

(c) Nonvolatile Silicones

Nonvolatile silicones, both water soluble and water insoluble, are also suitable as the oil component. Such silicones preferably have a viscosity of 10 to 600,000 centistokes, preferably 20 to 100,000 centistokes at 25° C. Suitable water insoluble silicones include amodimethicone, bisphenylhexamethicone, dimethicone, hexadecyl methicone, methicone, phenyl trimethicone, simethicone, dimethylhydrogensiloxane, stearoxytrimethylsilane, vinyldimethicone, and mixtures thereof. Also suitable are water soluble silicones such as dimethicone copolyol, dimethiconol, and the like. Such silicones are available from Dow Corning as the 3225C formulation aid, Dow 190 and 193 fluids, or similar products marketed by Goldschmidt under the ABIL tradename.

(d) Esters

Other nonvolatile oils include esters of the formula RCO—OR′ wherein R and R′ are each independently a C₁₋₂₅, preferably a C₄₋₂₀ straight or branched chain alkyl alkenyl or alkoxycarbonylalkyl or alkylcarbonyloxyalkyl.

(e) Glyceryl Esters of Fatty Acids

Also suitable are naturally occuring glyceryl esters of fatty acids, or triglycerides as well as synthetic or semi-synthetic glyceryl esters.

(f) Nonvolatile Hydrocarbons

Also suitable for use are nonvolatile hydrocarbons such as isoparaffins, hydrogenated polyisobutene, mineral oil, squalene, and so on.

2. Nonionic Surfactants

If desired, the aqueous developer composition may contain one or more nonionic surfactants. Recommended ranges are 0.01-10%, preferably 0.05-8%, more preferably 0.1-7% by weight of the total composition.

(a) Alkoxylated Alcohols

Suitable nonionic surfactants include alkoxylated alcohols, or ethers, formed by the reaction of an alcohol with an alkylene oxide, usually ethylene or propylene oxide. Preferably the alcohol is a fatty alcohol having 6 to 30 carbon atoms, and a straight or branched, saturated or unsaturated carbon chain. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Particularly preferred is Ceteareth 20, which is the reaction product of a mixture of cetyl and stearyl alcohol with ethylene oxide, and the number of repeating ethylene oxide units in the molecule is 20.

(b) Alkoxylated Carboxylic Acids

Also suitable as the nonionic surfactant are alkyoxylated carboxylic acids, which are formed by the reaction of a carboxylic acid with an alkylene oxide or with a polymeric ether. The resulting products have the general formula:

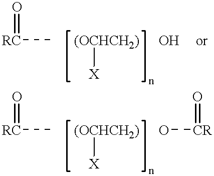

where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

(c) Sorbitan Derivatives

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

3. Thickening Agents

The aqueous developer composition may also comprise a thickening agent, if desired. Preferably, the amount of thickening agent is 0.001-5%, preferably 0.005-4%, more preferably 0.005-3% by weight of the total composition.

(a) Acrylic Copolymer Thickeners

One type of thickening agent for use in the developer composition is an acrylic polymer comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof. Preferably, the A monomer comprises one or more of acrylic acid or methacrylic acid, and the B monomer comprises is selected from the group consisting of a C₁₋₁₀, most preferably C₁₋₄alkyl acrylate, a C₁₋₁₀, most preferably C₁₋₄ alkyl methacrylate, and mixtures thereof Most preferably the B monomer is one or more of methyl or ethyl acrylate or methacrylate. Most preferably, the acrylic copolymer is supplied in an aqueous solution having a solids content ranging from about 10-60%, preferably 20-50%, more preferably 25-45% by weight of the polymer, with the remainder water. Preferably, the thickening agent is a polymer comprised of A, B, and C monomers wherein A and B are as defined above, and C has the general formula:

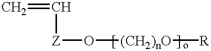

Preferably, in the copolymer used for the secondary thickening agent in the preferred embodiment of the invention, A and B are as above defined; and in the C monomer Z is (CH₂)ₘ, m is 1-2, n is 2, and o is 2-100, and R is a C₁₂₋₂₂ straight or branched chain alkyl. More preferably in the C monomer m is 1, n is 2, o is 10, and R is C₁₈ or stearyl, and the compound is steareth-10 allyl ether/acrylate copolymer, which may be purchased from Allied Colloids under the tradename Salcare SC90.

C. The Aqueous Based Hair Colorant

The aqueous based hair colorant composition contains one or more cationic dye molecules in aqueous medium. Preferably, the aqueous based colorant composition has a pH of about 4 to 7, and may be in the form of a water-in-oil or oil-in-water emulsion. The colorant composition may additionally comprise one or more of a quaternary ammonium surfactant, protein derivative, silicone oil, and a nonionic, zwitterionic, or betaine surfactant.

1. Cationic Dye Compounds

The aqueous based hair colorant composition comprises about 0-10%, 0.001-8%, more preferably 0.01-7% of one or more cationic dye molecules. The term “cationic dye molecule” means any hair colorant compound, or dye, that has a positive charge. In general, suitable cationic dyes must be stable in the presence of both hydrogen peroxide and persulfate bleaching compounds, and at both acidic and basic pH. For example, the aqueous based colorant composition preferably has a pH of about 4 to 12. After combining with the bleach powder the pH rises to about 7 to 12. If present, the cationic dye suitable for use in the invention must be stable in both the acidic and basic pH ranges found in the colorant and the combined mixture of bleach powder, aqueous developer, and aqueous based colorant. The term “stable” means that the dye compound does not decompose or change color, or it may change color provided however, if it does change color it must still be capable of depositing color on the hair, upon exposure to these conditions. Cationic dyes which are suitable for use in the compositions of the invention are those which do not exhibit any color change when subjected to the following screening test:

About 0.4 to 0.6 grams of the test dye is added to a base colorant composition comprising, by weight of the total composition,

| | |
|---|---|
| Propylene glycol | 1.5 |
| Ethoxydiglycol | 1.5 |
| Potassium cocohydrolyzed collagen | 3.0 |
| Cocoamidopropyl betaine | 2.0 |
| Hydrolyzed wheat protein/hydrolyzed wheat starch | 1.00 |
| Dimethicone copolyol/dimethicone/cyclomethicone | 0.50 |
| Cetrimonium chloride | 0.50 |
| Water | QS. |

The above mixture is then combined with the bleach powder and aqueous developer mentioned in Example 1, in a ratio of 1 part bleach powder, 2 parts aqueous developer, and 2 parts aqueous based colorant composition. Part of this mixture is placed in a container at room temperature, and the other part in a container stored at 40° C. After thirty minutes, a fresh mixture is made up and compared with the color of the mixtures maintained for 30 minutes at room temperature and 40° C. If the mixture maintained at room temperature for 30 minutes and the mixture maintained at 40° C. for 30 minutes are both the same color as the freshly made up mixture the test cationic dye is stable and suitable for use in the method and compositions of the invention. On the other hand, if one or the other, or both, of the mixtures maintained for 30 minutes is not the same color as the freshly made up mixture, then the test cationic dye is not suitable for use in the method and compositions of the invention. Suitable cationic dyes include those from the azo, phenazine, and thiazine families. Other types of suitable dyes, generally referred to as “basic dyes”, may be suitable in the invention provided they pass the general test set forth herein. Particularly preferred is the azo family dye referred to as Basic Orange 1; a phenazine family dye referred to as Basic Red 2; and a thiazine family dye referred to as Basic Blue 9, and Basic Yellow 28. These dyes may be purchased from Jos. H. Lowenstein Sons under the tradenames Lowacryl Orange 1; Lowacryl Red 2 or Lowacryl Red 2 Concentrate; Lowacryl Blue 9; and Lowacryl Yellow 28.

2. Cationic Surfactants

Preferably the aqueous based colorant composition comprises 0.001-10%, more preferably 0.005-8%, most preferably 0.01-5% of a cationic surfactant. Suitable cationic surfactants include cationic polymers, quaternary ammonium salts or the salts of fatty amines.

(a) Quaternary Ammonium Compounds

Quaternary ammonium compounds, or salts, have the formula:

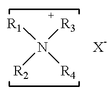

wherein R₁, R₂, R₃, and R₄ are each independently an aliphatic group of 1 to 22 carbon atoms, or aromatic, alkyl, aryl, or alkaryl group having 12 to 22 carbon atoms; with the proviso that there is at least one alkyl group having 12 to 22 carbon atoms. Preferably at least one of R₁, R₂, R₃, and R₄ are methyl while the remaining substituents are C₁₂-₂₂ aliphatic radicals. X is an anion selected from halogen, acetate, phosphate, nitrate and methyl sulfate, tosylate, and hydroxide radicals. The aliphatic groups may contain, in addition to carbon atoms, ether linkages as well as amido groups. Suitable quaternary ammonium compounds may be mono-long chain alkyl, di-long chain alkyl, tri-long chain alkyl, and the like. Examples of such quaternary ammonium salts include behenalkonium chloride, behentrimonium chloride, behentrimonium methosulfate, benzalkonium chloride, benzethonium chloride, benzyl triethyl ammonium chloride, cetalkonium chloride, cetrimonium chloride, cetrimonium bromide, cetrimonium methosulfate, cetrimonium tosylate, cetylpyridinium chloride, dibehenyl/diarachidyl dimonium chloride, dibehenyldimonium chloride, dibehenyldimonium methosulfate, dicapryl/dicaprylyl dimonium chloride, and the like.

Other quaternary ammonium salts useful as the cationic surfactant include compounds of the general formula:

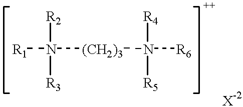

wherein at least one of R₁₆ is an aliphatic group having 16 to 22 carbon atoms, and the remaining R₁₋₆ are the same or different and are selected from alkyls having 1 to 4 carbon atoms and X is an anion as above defined.

(b) Amides

Amides which exhibit the general formulas set forth below are also suitable cationic surfactants:

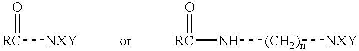

wherein R is a straight or branched chain saturated or unsaturated alkyl having 6 to 30 carbon atoms, n is an integer from 1 to 4, and X and Y are each independently H, or C₁₋₆ alkyl.

Preferred is an amide of the formula:

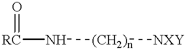

wherein R is a C₁₂₋₂₂ straight or branched chain alkyl, n is an integer from 1 to 4, and X is lower alkyl, preferably methyl.

(c) Amidoamine Salts

Also suitable are amidoamine salts, which are the condensation products of fatty acids with a polyfunctional amines, for example, those having the formula RCONH(CH₂)ₙNR₁R₂ where RCO is a fatty acyl group such as stearoyl, R₁ and R₂ are methyl or ethyl, and n is 2 or 3. Examples of such compounds include stearamidopropyl dimethylamine. Particularly preferred are amidoamine compounds complexed with a mild dimer acid, such as di(behenamidopropyl dimethyl amine) dimer dilinoleate or di(linoleamidopropyl dimethyl amine) dimer linoleate. Both ingredients are sold by Alzo, Inc. under the NECON tradename.

(d) Quaternary Imidazolinium Salts

Also, quaternary imidazolinium salts having the following general formula are suitable as the cationic surfactant:

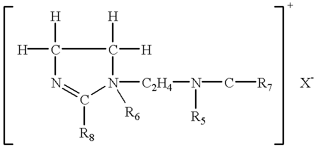

wherein R₅ is hydrogen or a C₁₋₄ alkyl; R₆ is a C₁₋₄ alkyl; R₇ is a C₈₋₂₂ alkyl; and R₈ is hydrogen, or a C₁₋₂₂ alkyl; and X is an anion as defined above.

(e) Amine Salts

Also suitable as the cationic surfactant are salts of fatty primary, secondary, or tertiary amines, wherein the substituted groups have 12 to 22 carbon atoms. Examples of such amines include dimethyl stearamine, dimethyl soyamine, stearylamine, myristylamine, tridecylamine, ethyl stearamine, and so on.

(f) Cationic Polymers

Also suitable as the cationic surfactant are cationic polymers such as:

(i) Quaternary Derivatives of Cellulose Ethers or Guar Derivatives

Examples of quaternary derivatives of cellulose ethers are polymers sold under the tradename JR-125, JR-400, JR-30M. Suitable guar derivatives include guar hydroxypropyl trimonium chloride.

(ii) Copolymers of Vinylpyrrolidone

Copolymers of vinylpyrrolidone having monomer units of the formula:

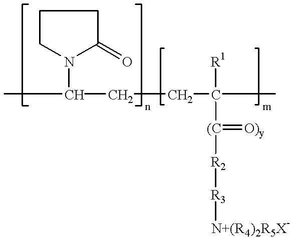

wherein R¹ is hydrogen or methyl, preferably methyl;

y is 0 or 1, preferably 1

R² is 0 or NH, preferably NH;

R³ is CₓH₂ₓ where x is 2 to 18, or —CH₂CHOH—CH₂, preferably CₓH₂ₓ where x

is 2;

R⁴ is methyl, ethyl, phenyl, or C₁₋₄ substituted phenyl, preferably methyl; and

R⁵ is methyl or ethyl, preferably methyl.

(iii) Polymers of Dimethyldiallylammonium Chloride

Homopolymers of dimethyldiallylammonium chloride, or copolymers of dimethyldiallylammonium chloride and acrylamide are also suitable. Such compounds are sold under the tradename MERQUAT by Calgon.

(iv) Acrylic or Methacrylic Acid Polymers

Homopolymers or copolymers derived from acrylic or methacrylic acid, selected from monomer units acrylamide, methylacrylamide, diacetone-acrylamide, acrylamide or methacrylamide substituted on the nitrogen by lower alkyl, alkyl esters of acrylic acid and methacrylic acid, vinylpyrrolidone, or vinyl esters are suitable for use.

(v) Cationic Silicones

As used herein, the term “cationic silicone” means any silicone polymer or oligomer having a silicon backbone, including polysiloxanes, having a positive charge on the silicone structure itself Cationic silicones that may be used in the compositions of the invention include those corresponding to the following formula, where the ratio of D to T units, if present, are greater than about 80 D units to 1 T unit:

(R)ₐG₃₋ₐ—Si—(—OSiG₂)ₙ—(—OSiG_{b}(R₁)_{2-6b})ₘ—O—SiG₃₋ₐ(R₁)ₐ

in which G is selected from the group consisting of H, phenyl, OH, C₁₋₁₀ alkyl, and is preferably CH₃; and a is 0 or an integer from 1 to 3, and is preferably 0; b is 0 or 1, preferably 1; the sum n+m is a number from 1 to 2,000 and is preferably 50 to 150; n is a number from 0 to 2000, and is preferably 50 to 150; and m is an integer from 1 to 2000, and is preferably 1 to 10; R is a C₁₋₁₀ alkyl, and R₁ is a monovalent radical of the formula C_{q}H_{2q}L in which q is an integer from 2 to 8 and L is selected from the groups:

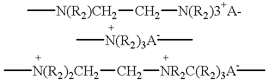

in which R₂ is selected from the group consisting of H, phenyl, benzyl, a saturated hydrocarbon radical, and is preferably an alkyl radical containing 1-20 carbon atoms; and A— is a halide, methylsulfate, or tosylate ion.

(vi) Polymeric Quaternary Ammonium Salts

Also suitable are polymeric quaternary ammonium salts such as Polyquaternium 31, 33, 34, 35, 36, 37, and 39.

(vii) Diquaternary Polydimethylsiloxanes

Also suitable are diquaternary polydimethylsiloxanes such as Quaternium-80, sold by Goldschmidt Corporation under the tradename ABIL-Quat 3272.

Examples of other cationic polymers that can be used in the compositions of the invention are disclosed in U.S. Pat. Nos. 5,240,450 and 5,573,709, which are hereby incorporated by reference.

The preferred aqueous based colorant compositions of the invention contain 0.001-10% by weight of a cationic surfactant which is a quaternary ammonium salt having the formula:

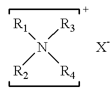

wherein R₁ is an aliphatic group of 1 to 22 carbon atoms, or aromatic, aryl, or alkaryl group having 12 to 22 carbon atoms; R₂ is an aliphatic group having 1-22 carbon atoms; R₃ and R4 are each alkyl groups of from 1 to 3 carbon atoms, and X is an anion selected from halogen, acetate, phosphate, nitrate, methyl sulfate, tosylate, and hydroxide radicals; preferably cetrimonium chloride.

3. Oily Ingredients

The aqueous based colorant composition may additionally comprise one or more oils, as described above with respect to the aqueous developer composition. Preferably the aqueous based colorant composition comprises 0.001-20%, more preferably 0.005-15%, most preferably 0.01-10% by weight of the total composition of one or more oils. The colorant may be in the form of a water-in-oil or oil-in-water emulsion form. Preferably the oils are either volatile or nonvolatile silicones as discussed herein with respect to the aqueous developer. Particularly preferred are silicones such as dimethicone copolyol, dimethicone, and cyclomethicone.

4. Humectants

It may be desireable to incorporate one or more humectants into the aqueous colorant composition. Preferably the colorant composition comprises 0.01-10%, more preferably 0.05-8%, most preferably 0.1-5% by weight of the total composition of humectant. Suitable humectants include monomeric, homopolymeric, and/or block copolymeric ethers as well as mono-, di-, or polyhydric alcohols.

Suitable ethers are formed by the polymerization of monomeric alkylene oxides, generally ethylene or propylene oxide. Such polymeric ethers have the following general formula:

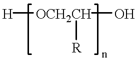

wherein R is H or lower alkyl and n is the number of repeating monomer units, and ranges from 1 to 500.

Also suitable are polyols such as glycerine or C₁₋₄ alkylene glycols and the like. Particularly preferred are C₁₋₄ alkylene glycols, in particular propylene and/or butylene glycol and ethoxydiglycol.

5. Anionic, Zwitterionic or Betaine Surfactants

If desired, the aqueous colorant composition may comprise one or more of an anionic, zwitterionic, or betaine surfactant, in the range of about 0.01-15%, preferably 0.05-10%, more preferably 0.1-8% by weight of the total composition.

(a) Anionic Surfactants

Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:

R₁—SO₃—M

wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones which have been formed in the reaction are hydrolyzed to give the corresponding hydroxy-alkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula:

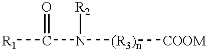

wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms. Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

(b) Amphoteric Surfactants

Suitable amphoteric surfactants may be imidazolinium compounds having the general formula:

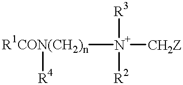

wherein R¹ is C₈₋₂₂ alkyl or alkenyl, preferably C₁₂₋₁₆; R² is hydrogen or CH₂CO₂M, R³ is CH₂CH₂OH or CH₂CH₂OCH₂CHCOOM; R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium. cation. Examples of such materials are marketed under the tradename MIRANOL, by Miranol, Inc.

Also suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants includ aminoalkanoates of the formula

R—NH(CH₂)ₙCOOM

or iminodialkanoates of the formula:

R—N[(CH₂)ₘCOOM]₂

and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof

(c) Zwitterionic Surfactants

Zwitterionic surfactants are also suitable for use in the compositions of the invention. The general formula for such surfactants is:

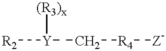

wherein R₂ contains an alkyl, alkenyl or hydroxy alkyl radical of from about 8 to about 18 carbon atoms, from 0 to about 10 ethylene oxide moieties and 0 or 1 glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R₄ is an alkylene or hydroxyalkylene of from about 1 to about 4 carbon atoms, and Z is a radical selected from the group consisting of carboxylate, sulfonate, sulfate, phosphonate, and phosphate groups.

Zwitterionics include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof Also suitable are sulfo- and amido- betaines such as coco dimethyl sulfopropyl betaine, cocamidopropyl betaine, stearyl dimethyl sulfopropyl betaine, and the like. Particularly preferred for use in the aqueous colorant composition is a betaine surfactant, in particular, cocamidopropyl betaine.

6. Protein Derivatives

It may be desired to incorporate one or more protein derivatives into the colorant composition to provide hair conditioning and moisturizing benefits in a range of about 0.01-15%, preferably 0.05-10%, more preferably 0.1-8% by weight of the total composition. The protein derivatives are generally formed by subjecting animal or vegetable proteins to enzymatic or chemical hydrolysis. If desired, the hydrolysate may be further reacted with other compounds to provide further derivatives. Examples of protein derivatives which are suitable for use in the compositions include hydrolysates of collagen, casein, albumen, egg protein, elastin, keratin, silk, soy protein, vegetable protein, wheat protein, wheat starch, wheat gluten, pea protein, oat protein, placental protein, and so on. Also suitable are derivatives of such hydrolysates, such as the reaction product of the hydrolysate with various fatty acids. Examples of such reaction products include potassium cocoyl hydrolyzed casein, collagen, corn protein, keratin, potato protein, rice bran protein, silk, marine collagen, and so on. Particularly preferred are hydrolysates of wheat protein and wheat starch and a potassium cocoyl derivative of hydrolyzed marine collagen which is referred to as potassium cocoyl hydrolyzed collagen.

II. THE METHOD

Immediately prior to use, the powder bleach composition, the aqueous developer, and the aqueous colorant composition are mixed to form the following composition:

(a) 1-20% inorganic persulfate,

(b) 1-20% hydrogen peroxide,

(c) 0-10% of at least one cationic dye molecule; and

(d) 0.01-20% of one or more cationic surfactants.

Preferably, the above composition is obtained by combining about 1-30% of the bleach powder, about 20-60% of the aqueous developer, and about 20-60% of the aqueous colorant composition, all percentages being by weight of the total mixture. More preferably, the mixture is made by combining about one part bleach powder, 2 parts aqueous developer, and 2 parts aqueous colorant composition.

The mixture is prepared immediately prior to use and applied to the hair. Preferably the mixture is applied to select strands of hair. In this case, a streaking cap or something similar is applied to the hair and strands of hair are pulled through the holes in the cap. If desired, an overcap is then placed on the head for about 5 to 60, preferably 10-25 minutes, or until the product has had an adequate lighten the hair. The overcap is desireable because it causes retention of body heat, which will accelerate the coloration process. If desired, additional heat may be applied to the hair during the color process but it is not necessary. Subtle variations in tonality and hue of the hair are achieved with shorter exposure times at room temperature, e.g. will cause a “lifting” of the natural hair color. Subtle to pronouced variations in shade can be achieved with longer exposure times and, if desired, the application of heat for all or part of the exposure time. The hair is then rinsed well with water to remove the colorant. The hair is then either directly conditioned with a hair conditioner, or if desired, the hair is first shampooed and then treated with a conditioner. The colored hair has variations in tonality, hue and/or shade. In addition, the hair is soft and healthy, and does not exhibit the dry, damaged feel and appearance that often characterizes hair colored with oxidation dyes. While most semi-permanent colors are expected to last from six to twelve shampoos, most unexpectedly, the compositions of the invention can last as long as color provided by permanent, or oxidation dyes. Without being bound by this explanation, it is generally believed that when the bleach powder, aqueous developer, and aqueous colorant composition are mixed and applied to hair, the cationic dyes and surfactants bind to the negatively charged hair surfaces and cause increased retention of color, in other words color retention on the hair is longer when the hair is more damaged because of the increase in negatively charged sites on damaged hair. In addition, the addition of ethanolamine, in particular 0.01-25% monoethanolamine to the aqueous colorant composition which does not contain a cationic dye, enables a reduction in the amount of persulfate required to lighten hair, and causes the colored hair to be more conditioned.

The invention will be further described in connection with the following examples, which are set forth for the purposes of illustration of only.

### EXAMPLE 1

A powder composition, aqueous developer composition, and aqueous based colorant composition were prepared as follows:

| Powder Bleach Composition | w/w % |
|---|---|
| Potassium persulfate | 45.00 |
| Sodium persulfate | 5.00 |
| Sodium metasilicate | 11.50 |
| Silica | 2.00 |
| Hydrated silica | 2.00 |
| Sodium stearate | 10.67 |
| EDTA | 2.00 |
| Hydroxyethylcellulose | 3.09 |
| Sodium lauryl sulfate | 2.00 |
| Sodium chloride | 5.00 |
| Sucrose | 7.16 |
| Ultramarine blue | 0.08 |
| Sodium silicate | 4.50 |

The bleach composition was made by combining all of the ingredients and mixing well.

| Peroxide Developer | w/w % |
|---|---|
| Water | 74.69 |
| Methyl paraben | 0.05 |
| EDTA | 0.02 |
| Mineral oil | 0.60 |
| Cetearyl alcohol/ceteareth-20 (80:20) | 3.75 |
| Cetearyl alcohol | 0.80 |
| Cyclomethicone/trimethylsiloxysilicate (50:50) | 0.01 |
| Trimethylsilylamodimethicone/C11-15 pareth-7/ | 2.00 |
| C12-16 pareth-9/trideceth-12/glycerin/water | |
| (20:6:4:2:3:65) | |
| Disodium phosphate | 0.03 |
| Phosphoric acid | 0.04 |
| Hydrogen peroxide (35% aqueous solution) | 18.00 |
| Steareth-10 allyl ether/acrylates copolymer | 0.01 |

The composition was made by combining all of the ingredients and mixing well. The pH of the developer composition was about 3.8.

| Colorant Composition | w/w % |
|---|---|
| Propylene glycol | 1.5 |
| Ethoxydiglycol | 1.5 |
| Potassium cocohydrolyzed collagen | 3.0 |
| Cocoamidopropyl betaine | 2.0 |
| Hydrolyzed wheat protein/hydrolyzed wheat starch | 1.00 |
| Dimethicone copolyol/dimethicone/cyclomethicone | 0.50 |
| Cetrimonium chloride | 0.50 |
| Basic orange 1 | 1.00 |
| Basic Red 2 | 1.00 |
| Basic blue 9 | 1.00 |
| Water | QS |

The colorant composition was made by combining all ingredients and mixing well. The pH of the composition was about 5.

About 30 grams of the bleach composition, about 60 grams of the developer composition, and about 60 grams of the colorant composition were combined and mixed well in a bowl. A frosting cap having a plurality of small holes was put on the subject's hair. Strands of hair were pulled through the holes in the frosting cap. The composition was applied to the strands of hair and covered with an overcap and left for 15 to 25 minutes. The caps were removed from the hair and the hair was rinsed well with water. The hair was then shampooed and conditioned. The hair to which the composition was applied was both colored and highlighted, exhibiting variations in tonality and hue.

### EXAMPLE 2

An aqueous based colorant composition was prepared as follows:

| | w/w % |
|---|---|
| Water | 72.55 |
| Methylparaben | 0.20 |
| Propylene glycol | 2.00 |
| Ethoxydiglycol | 2.00 |
| Cocoamidopropyl betaine (35%) | 2.00 |
| Disodium EDTA | 0.05 |
| Hydroxyethylcellulose | 1.00 |
| Cetrimonium chloride (25%) | 0.50 |
| Potassium cocoyl hydrolyzed collagen (40%) | 1.50 |
| Diazolidinyl urea | 0.20 |
| Dimethicone copolyol | 0.50 |
| Hydrolyzed wheat protein/hydrolzed wheat starch | 0.50 |
| Wheat amino acids | 0.50 |
| Ethanolamine | 15.00 |

A developer composition was prepared as follows:

| | w/w % |
|---|---|
| Methyl paraben | 0.05 |
| EDTA | 0.02 |
| Mineral oil | 0.60 |
| Cetearyl alcohol/ceteareth-20 (80:20) | 4.10 |
| Lauryl pyrrolidone | 2.00 |
| Cyclomethicone/trimethylsiloxy silicate (50:50) | 0.01 |
| Trimethylsilylamodimethicone/C11-15 pareth-7/ | 1.25 |
| C12-16 pareth-9/trideceth-12/glycerin | |
| water (20:5:4:2:3:65) | |
| Disodium phosphate | 0.02 |
| Phosphoric acid | 0.02 |
| Hydrogen peroxide (35%) | 26.00 |
| Steareth-10 allyl ether/acrylates copolymer | 2.00 |
| Water | QS |

About 30 grams of the powder bleach composition of Example 1, about 60 grams of the developer composition, and about 60 grams of the colorant composition were combined and mixed well in a bowl. The composition had a pH of 11 to 11.5. A frosting cap having a plurality of small holes was put on the subject's hair. Strands of hair were pulled through the holes in the frosting cap. The composition was applied to the strands of hair and covered with an overcap and left for 15 to 25 minutes. The caps were removed from the hair and the hair was rinsed well with water. The hair was then shampooed and conditioned. The hair to which the composition was applied was both colored and highlighted, exhibiting variations in tonality, hue, and shade.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A single composition for simultaneously coloring and highlighting hair to provide hair fibers having variations in tonality, hue, and/or shade, comprising, by weight of the total composition:
(a) 1-20% inorganic persulfate,
(b) 1-20% hydrogen peroxide,
(c) 0-10% of at least one cationic dye molecules; and
(d) 0.01-20% of one or more cationic surfactants.

2. The composition of claim 1 wherein the inorganic persulfate is an alkali metal or alkaline earth metal persulfate, or mixtures thereof.

3. The composition of claim 1 wherein the cationic dye molecules are selected from the group consisting of azo, phenazine, thiazine, and mixtures thereof

4. The composition of claim 1 wherein the cationic surfactant comprises a quaternary ammonium compound.

5. A one step method for simultaneously coloring and highlighting hair to provide hair fibers having variations in tonality, hue, and/or shade comprising the steps of:
(a) combining, immediately prior to use, (I) a powder composition comprised of at least one alkali metal or alkaline earth metal persulfate, (ii) an aqueous developer composition comprised of hydrogen peroxide; and (iii) an aqueous based colorant composition; and
(b) applying the mixture of (a) to the hair for a period of time sufficient to cause coloration and highlighting of the hair.

6. The method of claim 5 wherein the powder composition comprises 15-65% by weight of the total composition of sodium or potassium persulfate, or mixtures thereof.

7. The method of claim 6 wherein the powder composition further comprises 5-60% by weight of the total composition of one or more particulate fillers.

8. The method of claim 7 wherein the powder composition further comprises 0.01-2% by weight of inorganic colorant.

9. The method of claim 5 wherein the aqueous developer composition comprises, by weight of the total composition, 50-99% water, 1-30% hydrogen peroxide, and 0.01-30% of an oily phase.

10. The method of claim 9 wherein the aqueous developer composition additionally comprises 0.01-10% of a film forming polymer.

11. The method of claim 5 wherein the aqueous based colorant composition comprises, by weight of the total composition, 0.01-10% of one or more cationic dye molecules.

12. The method of claim 11 wherein the cationic dye molecules are selected from the group consisting of azo, phenazine, thiazine, and mixtures thereof

13. The method of claim 12 wherein the aqueous based colorant has a pH of 4 to 7.

14. The method of claim 13 wherein the aqueous based colorant composition further comprises 0.01-20% of a cationic surfactant.

15. The method of claim 12 wherein the aqueous based colorant further comprises, by weight of the total composition, 0.01-300% of a silicone selected from the group consisting of volatile silicone, nonvolatile silicone, and mixtures thereof

16. The method of claim 15 wherein the aqueous based colorant composition further comprises 0.1-20% humectant.

17. The method of claim 12 wherein the aqueous based colorant composition further comprises 0.1-10% of one or more protein derivatives.

18. The method of claim 5 wherein the mixture of (a) comprises, by weight of the total mixture, about 1-30% (i); 20-60% of (ii); and 20-60% of (iii).

19. The method of claim 18 wherein the mixture of (a) has a pH of about 7.5 to 11.

20. The method of claim 19 wherein the mixture of (a) is applied to the hair for about 5 to 40 minutes and then rinsed out with water.
